(19) United States
(12) Patent Application Publication
(10) Pub. No.: US 2006/0212961 A1
(43) Pub. Date: Sep. 21, 2006

(54) Rice transposon gene

(76) Inventors: Shigeru Iida, Aichi (JP); Masahiko Maekawa, Okayama (JP); Kazuo Tsugane, Aichi (JP)

Correspondence Address: DRINKER BIDDLE & REATH (DC), 1500 K STREET, N.W., SUITE 1100, WASHINGTON, DC, 20005-1209, US

(21) Appl. No.: 10/563,051

(22) PCT Filed: Mar. 19, 2004
(86) PCT No.: PCT/JP04/03772

§ 371 Date: Apr. 13, 2006

(30) Foreign Application Priority Data
Jul. 4, 2003 (JP) 2003-270879

(51) Int. Cl. A01H 5/00; C12N 5/04; C12N 15/82; A01H 1/00

(52) U.S. Cl. 800/278; 435/468; 435/419; 800/320.2

(57) ABSTRACT

The present inventors identified the causative gene of pyl mutant, which is mutable and shows a lowered chlorophyll accumulation, and discovered that a new type of transposon classified to Ac/Ds type is involved in the mutation. It is confirmed for the first time that the Ac/Ds type transposon nDart (SEQ ID NO: 1) in rice has transposon activity under normal cultivation conditions. Furthermore, an autonomous element Dart was discovered by analyzing said Ac/Ds type transposon.

### FIELD OF THE INVENTION

The present invention relates to a rice transposon gene, more specifically to a rice Ac/Ds type transposon gene and it's autonomous element.

### PRIOR ART

Based on the type of the transposition, transposable elements are broadly classified into class I element, where a transposable element transposes via an RNA intermediate, and class II element, where a transposable element transposes as DNA molecule after cut out of DNA. A large scale rice gene tagging system has been developed using Tos17, a class I element, however, it has been known that highly frequent somatic mutations are induced, because Tos17 transposition accomplishes via a callus culture (Trend. Plant Sci. 6: 127-134 (2001)). Whilst, MITE-based mPing is reported as a class II element (Nature, vol. 421, No. 6919, pp. 167-170 (January 2003))

### PROBLEMS WHICH THIS INVENTION ATTEMPTS TO SOLVE

However, mPing transposition needs anther culture or γ-irradiation, which induces somatic mutation or highly frequent mutation (Nature, vol. 421, No. 6919, pp 167-170 (Jan. 2003)). Moreover, it is considered that only a part of possible mutations is obtainable because of the target sequence specificity of these transposons. Therefore, a new tagging system is required.

### MEANS FOR SOLVING THE ABOVE PROBLEMS

The present inventors identified the causative gene of pyl (pale-yellow leaf) mutation, which is mutable and lowers the chlorophyll accumulation, and found that a new transposon, classified to a Ac/Ds type element, is involved in the mutation. These results confirmed for the first time the presence of Ac/Ds type transposon nDart (nonautonomous Ds-related active rice transposon), which has a transposon activity under the normal cultivation conditions of rice. Furthermore, the inventors discovered the autonomous element Dart during the analysis of the Ac/Ds type transposon.

Therefore, the present invention is a rice transposon gene (nDart) comprising DNA of the following (1) or (2);

(1) DNA comprising the nucleotide sequence of SEQ ID NO: 1.

(2) DNA comprising the nucleotide sequence, which is more than 98%

homologous to the nucleotide sequence of (1), wherein said DNA transposes by subjecting rice containing said DNA to the treatment with a chemical agent.

Furthermore, the present invention is a rice transposon gene (Dart) comprising DNA of the following (3) or (4);

(3) DNA comprising any nucleotide sequence of SEQ ID NOs: 6-8.

(4) DNA comprising the nucleotide sequence, which is more than 98%

homologous to the nucleotide sequence of (3), wherein said DNA transposes by subjecting rice containing said DNA to the treatment with a chemical agent.

It has been known that the transposition frequency of a transposon increased by subjecting a plant to culture under the condition to induce active cell division. Therefore, for the increase of the transposition frequency of nDart and Dart, which transpose under normal cultivation conditions, furthermore, it is effective for plants to subject to grow under stressful conditions. The stressful conditions include the treatment with a chemical agent, 5-azacytidine which induce DNA demethylation, the exposure to radiation, such as UV and γ-rays, or the application of an artificial culture system, such as culture of callus, which is obtained by dedifferentiation of plant cells. Said treatments increase the transposition frequency of nDart and Dart and an increase in the mutation frequency enables to obtain efficiently the desired mutants.

Moreover, the present invention is a plasmid containing any transposon gene above described. The useful plasmids here include Ti plasmid and binary vectors such as pBI-121 plasmid etc. The useful promoters here include ³⁵S promoter of cauliflower mosaic virus, heat shock promoter, chemotaxis promoter and others. In addition, there are no restrictions on the method of ligation between promoters and said gene, and general method of genetic engineering could be applied arbitrarily.

Also, the present invention is a transformant, wherein any one of said transposon gene is transduced. The host is preferably a plant and preferably Arabidopsis, tobacco, tomato, petunia, crucifer, cotton plant or maize. To transform the plant, using a general method of genetic engineering, these genes can be inserted into said plasmid and the plant can be transformed.

Also, the present invention is a transformed plants or seed, wherein any one of said transposon gene is transduced by any one of said methods. The plant is preferably Arabidopsis, tobacco, tomato, petunia, crucifer, cotton plant or maize.

### BRIEF DESCRIPTION OF THE DRAWINGS

FIG. 1 shows a mutable pyl-v mutant.

FIG. 2 shows pyl-stb (left), whose phenotype is stable and wild type “Taichung No. 65” (right). An individual piece at 7 days after seeding on modified white media is shown.

FIG. 3 shows ply gene map-based cloning.

FIG. 4 shows the result of a gel electrophoresis of PCR product of Example 1.

FIG. 5 shows OsClpP gene (a ply mutant). A black square shows an exon and atg shows the initiation codon.

FIG. 6 shows the flanking regions of the transcription initiation site of OsClpP gene in a ply-stb mutant. The small letters show the region to be translated to a protein and atg with an underline shows the initiation codon. The first arrow show the transcription initiation site of a wild type and other arrows show transcription initiation sites of ply-stb mutants. The numbers on a shoulder show the numbers of cloning.

FIG. 7 shows the structure (A) of OsClpP gene of a revertant and the footprint (B) remained. The atg with an underline shows an initiation codon.

FIG. 8 shows an autonomous element searched by BLAST.

FIG. 9 shows the result of a gel electrophoresis of DNA of a mutated region of intercrosses (B3F1) between indica rice (Kasarasu) and pyl-v mutants of Taichung No. 65 (T-65). (1) shows the nDart1-0 (chromosome No. 3) mutation, (2) nDart1-4(3-1) (chromosome No. 3), (3) nDart1-12 (chromosome No. 1), (4) nDart200-2 (chromosome No. 12). Line 1 shows Kasarasu, Line 2 shows T-65, pyl-v and Line 3-21 shows intercrosses (B3F1).

### DETAILED DESCRIPTION OF THE INVENTION

The inventors searched transposons with highly homologous to nDart using BLAST (Basic Local Alignment Search Tool). The database sites of the search are US Biotechnology information center and DNA data bank of National Institute of Genetics. The Query was nDart (SEQ ID NO: 1). RiceGAAS system (Yu. J, Hu. S, (2002) Science 296: 79-92) was used as a gene prediction program. The result of our analysis using BLAST is shown in Table 1.

Thirty-one rice nucleotide sequences are homologous to nDart and 7 out of them are more than 98% homologous to nDart.

Any cultivar could carry an active DNA transposon as an efficient mutagen by crossing without an exogeneous gene for breed improvement, since the nucleotide sequence (SQE ID NO: 1) of nDart has been determined in the present invention.

By the transposition of nDart it is possible to obtain a mutant with disrupted gene and to isolate a stable mutant, wherein nDart re-transposed and a mutated footprint remained, and a revertant, wherein a mutated phenotype reverted to a wild one, from self-fertilized progenies of the mutants. It is possible to identify the gene disrupted by re-insertion of transposed nDart, by way of designing primers inside nDart sequence, performing reverse PCR and others. By analyzing the relation between these mutants and their genes, the function of the genes can be understood. In this case, the gene disrupted by a transposed nDart can be identified using said method.

Furthermore, it is possible to obtain a mutant with disrupted gene by transduction of the transposon to other plants by general transformation method. By analyzing the relation between these mutants and their genes, the function of the genes can be investigated. In this case, the gene disrupted by the transposed transposon can be identified using said method.

As a way to utilize nDart and Dart of the present invention, a tagging system with a transposon for rice or other desired plants can be produced by using these transposons as mutagens. In the case of producing a tagging system for rice particularly, an intercross easily enables any rice cultivar to carry nDart and active Dart genes. Since said tagging system dose not contain an exogeneous gene, it is possible to develop the tagging system in a large scale on an outside field, using a general cultivation method without using a physical containment facility, which is necessary for the cultivation of a genetically modified plant. The mutants thus obtained could be analyzed by a method of genetic or reverse genetic analysis. The genetic method is a method to isolate a causative gene based on the phenotype of a mutant and enables to identify and to isolate the causative gene easily using a tag (a transposon), since the transposon of the present invention and the phenotype of a mutant are linked.

Also, the reverse genetic analysis is a method of isolating mutants, wherein a certain function of a gene is lost out of the original genes. Firstly, DNA pool is prepared after isolation of DNA from various kinds of mutants. The pool was PCR screened and the screening enables to pick up a mutant, wherein a transposon is inserted to the expected gene.

### EXAMPLES

The following Examples illustrate this invention, however, it is not intended to limit the scope of the invention.

### Test Example 1

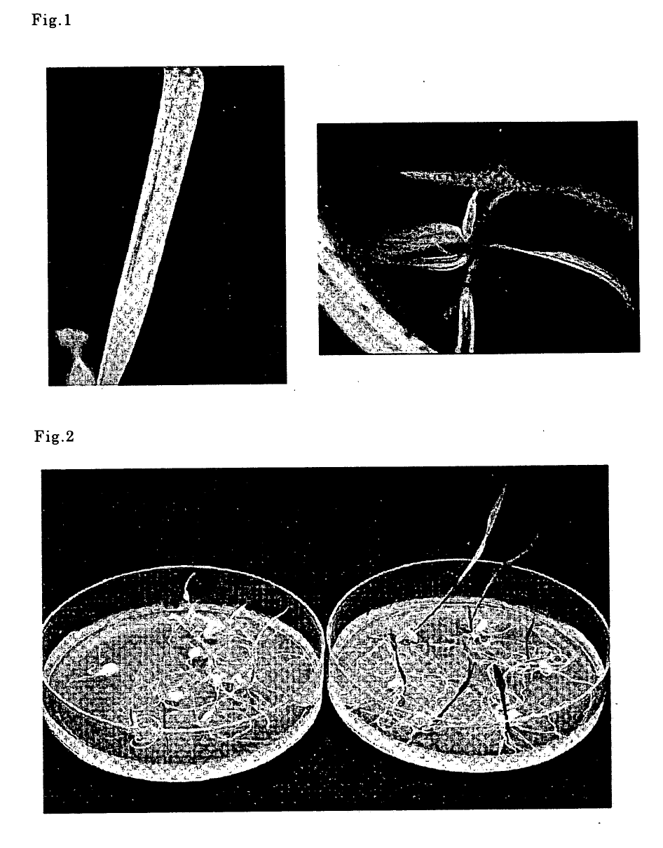

Maekawa, a co-inventor of the present invention, isolated a mutable pyl-v mutant (FIG. 1), which is generated by intercross (F2) between japonica rice H-126 and indica rice C-5052 and has a variegation with a lowered chlorophyll accumulation in 1986 (FIG. 1). Mutability is expected to be due to the insertion and release of a DNA-type transposon based on the observation that, in pyl-v mutants, a dark green-colored sector, similar to wild type, appears along with a cell group in a corn-colored leaf, as well as on the genetic analysis.

### Test Example 2

Then, pyl-stb (pale-yellow leaf-stable) is isolated, wherein the pyl phenotype is apparently stable, after preparing a near-isogenic lines by repeated intercross between a pyl-v mutant obtained in Test Example 1 and japonica rice “Shiokari” or “Taichung No. 65”.

Since pyl-stb (pale-yellow leaf-stable) leads to isolation again of a mutable line by an intercross and turns into a mutable line by the treatment with a chemical agent, pyl-stb line is transiently stable owing to separation or inactivation of an autonomous element.

A pyl-stb mutant seedling germinated on the soil is blighted before the fourth leaf stage. In this Example, pyl-stb seed was cultured on an agar media under sterile condition until the fourth leaf stage and then transplanted on the soil. In this way, pyl-stb resulted in fruitage with high probability. A pyl-stb seed can grow over the fourth leaf stage, if it is seeded and germinated under white fluorescent lamp with continuous light at 26 μmol m⁻² sec⁻¹, 28° C. and sterile condition with modified white medium (Kusumi K., Mizutani A. et al. (1997) Plant J. 12: 1241-50). The pyl-stb grown over the fourth leaf stage can come to fruition with high probability after transplanted to the soil. FIG. 2 shows the growth of pyl-stb seedlings.

### Example 1

In this Example, a map-based cloning was conducted in order to identify a DNA-type transposon gene, which induce variegation of pyl-v, and the causative gene of pyl mutation.

For the analysis of a map-based cloning, F2 population was used, which is generated by intercross between indica rice “Kasarasu” and near-isogenic lines, which are grown after backcross between pyl-v mutant obtained in Example 1 and “Shiokari”. DNA were isolated from 21 pyl-v mutants which were derived from this F2 seedlings and a simple mapping of pyl was conducted by using 54 landmark SSR marker (Theor. Appl. Genet. 100:697-712 (2000)) covering 12 chromosomes. As the result, it was found that pyl gene is located within 22cM between RM282 and RM251 of the chromosome No. 3 short arm.

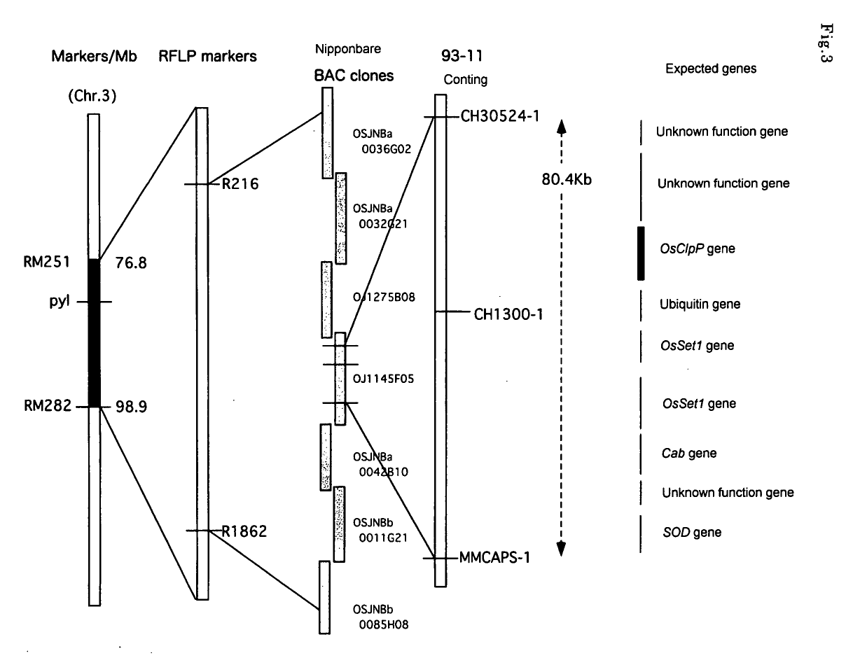

Then, EST clone (Plant cell 14, 525-35 (2002)) of Nipponbare located between the above 2 markers was selected. Based on the nucleotide sequence of these EST clones, a conting (Science 296: 79-92 (2002)) was selected, which is a group of linked genome DNA sequence of indica-type 93-11 containing these EST clones. At the same time, BAC clone of Nipponbare, reported by CSHL group in USA, was selected and 18 markers were prepared, which are usable as primers to get PCR product for identification, located straddling the DNA region, where there are more than 8 bp difference between the nucleotide sequences of the two selected clones. Using these markers, those seedlings expressing pyl phenotype out of 11800 F2 seedlings were selected and 3112 genetically modified seedlings were obtained. As the result, the candidate genes of pyl within about 80.4 kb were obtained. FIG. 3 shows the mapping diagram.

9 ORF were expected in this region, designed primer sets to amplify all these ORF regions of the genome and examined the PCR products of “Taichung No. 65”, pyl-stb and “Kasarasu”.

PCR reaction mixture (50 μl) contains 2.5U LATaq, lxGC buffer, 400 μM DATP, 400 μM dGTP, 400 μM dCTP, 400 μM dTTP (Takara), 0.2 μM primer set, 100 ng genomic DNA of “Taichung No. 65”, pyl-stb and “Kasarasu”, and sterilized MilliQ water (Milipore) for the volume adjustment. The PCR products were separated by 0.8% LOIII agarose gel (Takara) electrophoresis.

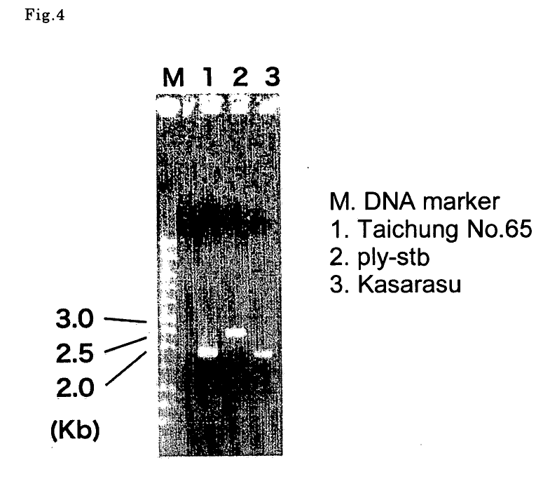

The PCR, with primers Clp-3F (SEQ ID NO: 2) and Clp-4R (SEQ ID: 3), resulted in about 600 bp difference between the products of pyl-stb and other lines. FIG. 4 shows the result of the electrophoresis.

The gene amplified by the primers Clp-3F and Clp-4R is a gene (OsClpP, SEQ ID No: 9), which is 80% homologous to ClpP5 gene (Trend. Plant Sci. 6:127-134 (2001) of Arabidopsis and is probably coding a protease transported to chloroplast.

### Example 2

In this Example, the inventors determined nucleotide sequence of PCR products to confirm the difference of PCR products amplified in Example 1.

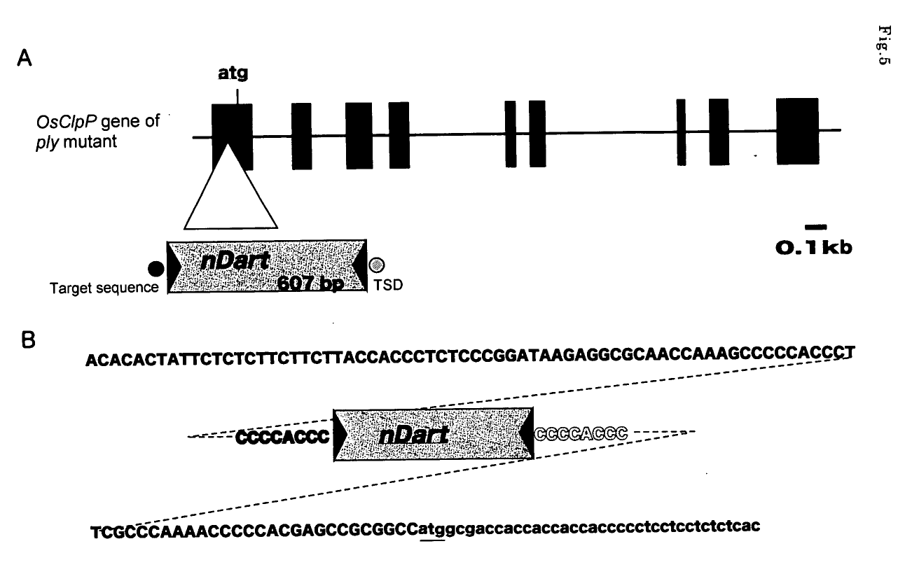

the PCR products of Example 1 were purified by QIA quick PCR purification Kit (Qiagen), determined the nucleotide sequences by the sequencer (ABI PRISM377, Applied Biosystem) and analyzed the sequences by various softwares. Then, it was found that a 607 bp sequence (SEQ ID NO: 1) is inserted to the exon 1 of OsClpP gene. FIG. 5 shows the structure of the insertion site.

Eight bp TSD (Target Site Duplication) of the target sequence is induced at the time of DNA transposon insertion in this region and 19 bp TIR (Terminal Inverted Repeat) is generated at both ends of the 607 bp insert.

This insert sequence is classified to Ac/Ds type based on 8 bp TSD and the similarity of TIR to known DNA transposon. Table 2 shows the comparison between the known Ac/Ds type transposon and this insert sequence (nDart). This insert sequence is a new type transposon gene similar to Ds without accompaniment of an autonomous element.

### Example 3

To determine the transcription initiation site and whole length of the gene, which is expected to code OsClpP protein and contains insert nDart (SEQ ID NO: 1) in a pyl mutant, in this Example, the inventors performed 5′RACE and 3′RACE to notice CAP structure of mRNA.

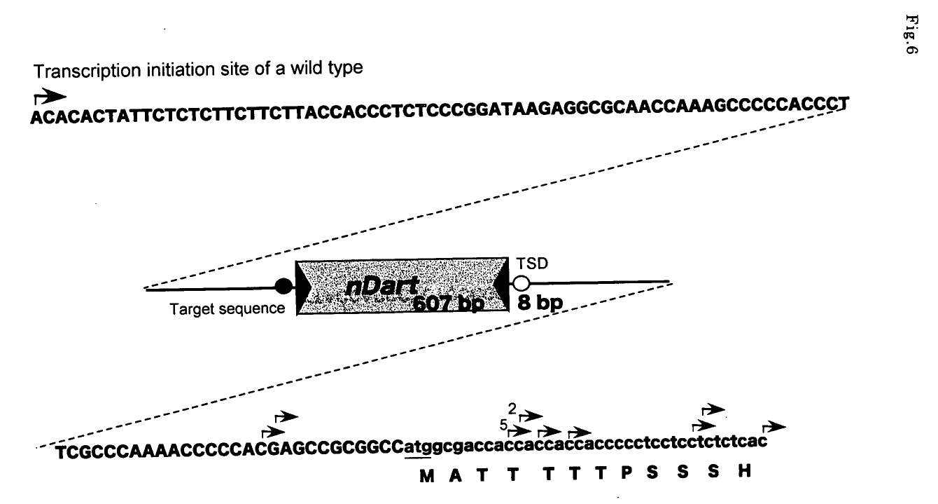

RNA was isolated from pyl-stb using guanidinium thiocyanate HCl and prepared cDNA from 1 μg total RNA using THERMOSCRIPT (Invitrogen). The transcription initiation site was determined by repeated PCR using the primer PG8-813R (SEQ ID NO: 4) and the primer Clp-3R (SEQ ID NO: 5), prepared on the basis of GeneRacer Kit (Iinvitrogen) and the nucleotide sequence of OsClpP gene, and using said cDNA as a template. FIG. 6 shows the result.

It was found that most of the transcription initiation sites are located at the downstream of the sequence coding methionine, which is the first amino acid in the translated protein, in pyl-stb and thought that pyl mutation is due to OsClpP gene.

### Example 4

To examine the release of nDart in 49 revertants derived from 15 independent lines appeared from pyl-v mutants, in this Example, the inventors performed PCR using primers Clp-3F (SEQ ID NO: 3) and Clp-4R (SEQ ID NO: 4) to amplify the region from the first exon to the seventh exon of OsClpP gene.

PCR reaction mixture (50 μl) contains 2.5U LATaq, 1×GC buffer, 400 μM dATP, 400 μM dGTP, 400 μM dCTP, 400 μM dTTP (Takara), 0.2 μM primer set, 100 ng genomic DNA of a revertant, and sterilized MilliQ water (Milipore) for the volume adjustment. The PCR products was separated by 0.8% LOIII agarose gel (Takara) electrophoresis and confirmed that PCR products with the same size to that of the DNA fragment, wherein the transposon gene (SEQ ID NO: 1) is deleted, was amplified.

### Example 5

To confirm that the PCR products amplified in Example 4 was the result of transposition of the transposon gene (SEQ ID NO: 1) from the first intron region of OsClpP gene, in this Example, the inventors determined the nucleotide sequence of PCR products.

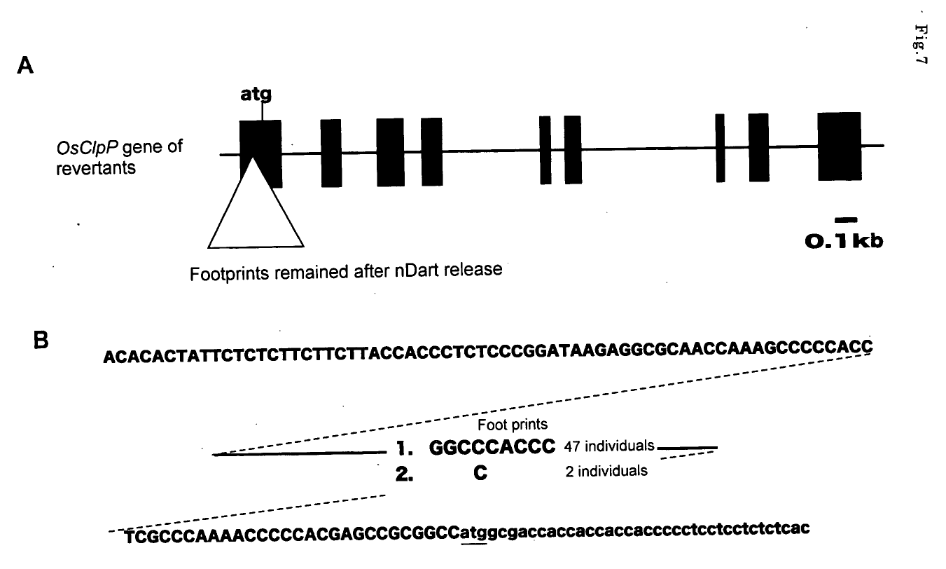

The PCR products of Example 4 were purified by QIA quick PCR purification Kit (Qiagen), determined the nucleotide sequences by the sequencer (ABI PRISM377, Applied Biosystem) and analyzed the sequence using various softwares. FIG. 7 shows the results.

Any change on the exon 1 of OsClpP gene was not observed except the change of the nucleotide sequence by two kinds of footprint in said revertants. Also, the change by said footprint does not influence on the translation from mRNA of OsClpP to a protein and the same CLP protein to wild type might be produced in said revertants.

### Example 6

In this Example, it was confirmed that pyl-stb seeds obtained in Test Example 2 were able to be changed to a pyl-v mutant by the treatment with azacytidine treatment.

pyl-stb seeds were macerated in 0.15, 0.3 or 0.45 mM azacytidine aqueous solution for 24 hrs at 30° C., washed them, left for germination and examined the expression of pyl-v. Table 3 shows the result. The frequency of the transposition of nDart increased as the concentration of azacytidine increased.

### Example 7

In this Example, autonomous element transposases controlling the transposition of nDart was searched by Blast (Basic Local Alignment Search Tool).

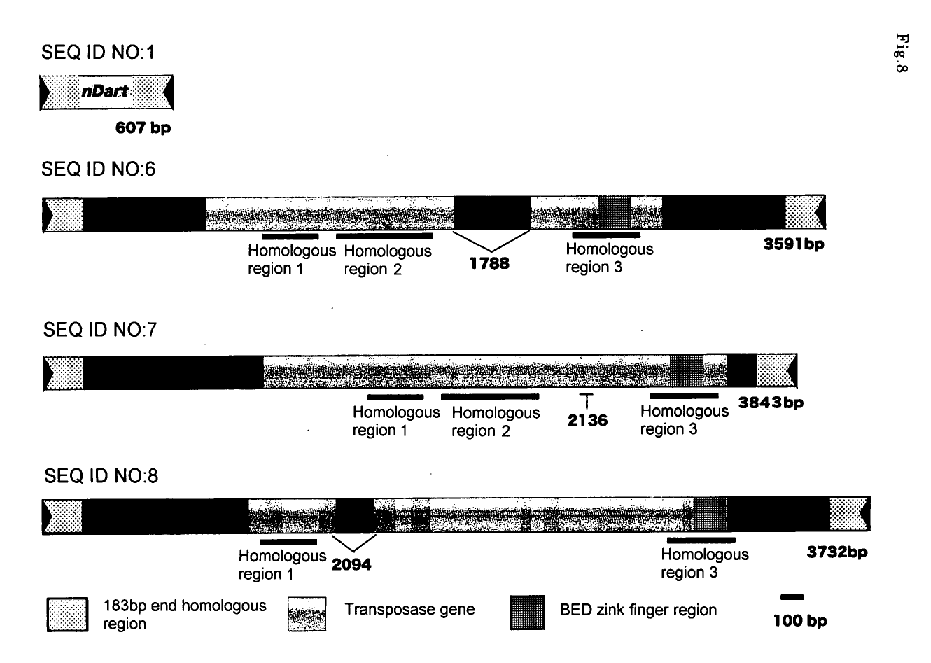

Since it was expected that a nucleotide sequence containing a transposase gene has the same sequence to that of nDart at both ends of the sequence and has transposase gene inside the sequence, the sequence having the same sequence of both ends of nDart (SEQ ID NO: 1) was searched and 3 sequences were found (SEQ ID NOs: 6-8). FIG. 8 shows the sequences.

The sequence of SEQ ID NO: 6 has the end sequence with the highest homology among them. Each 183 bp of the both ends of SEQ ID NO: 6 is more than 98% homologous to the both ends of SEQ ID NO: 1 and it is shown by a gene prediction program that the ORF inside the sequence of SEQ ID NO: 6 has transposase genes. It was thought that the sequence of SEQ ID NO: 6 was an autonomous element, which is highly homologous to Tam3 transposase reported for Antirrhinum.

Then, an autonomous element having the highest homology to Tam3 predicted from the sequence of SEQ ID NO: 6 was searched. Since the transposase gene of Tam3 is reported to have a structure without introns, transposase genes without introns were searched from said 31 nucleotide sequences and the sequence of SEQ ID NO: 7 was identified. Based on the analysis by a gene prediction program, the sequence of SEQ ID NO: 7 has a structure without introns and contains BED Zinc finger region, a DNA binding region, at the 3′ end. The sequence of SEQ ID NO: 7 may be an autonomous element controlling the transposition of nDart.

Based on the transposase gene predicted from the sequence of SEQ ID NO: 7 as an index, nucleotide sequences with different splicing patterns were searched and the sequence of SEQ ID NO: 8 was identified. As shown in FIG. 8, the sequence of SEQ ID NO: 8 has homologous regions 1 and 3 but not 2 and a different expression pattern and function from that of SEQ ID NO: 7 is expected.

### Example 8

Indica rice (Kasarasu) and pyl-v mutants of Taichung No. 65 (T-65) were intercrossed, obtained in Test Example 2, and nDart and an autonomous element (Dart) were transduced into Kasarasu. The backcross was performed for 3 times, and theoretically, the frequency of transformation to Kasarasu genotype is expected to be 93.8%.

In the case of pyl mutant, the causative gene of the mutation is nDart1-Origin (referred to as nDart1-0) inserted to OsClpP gene. By BLAST analysis, it was found that japonica rice (Nipponbare) has at least 14 nDarts, which are more than 98% homologous to nDart1-0. The nDart was named as nDart1-1 to nDart1-12, based on the degree of homology to nDart1-0. Since two sets of nDart (nDart1-3 and nDart1-4) has the same nucleotide sequence but locates on different loci of rice genome, they are classified according to the chromosome number shown in parentheses.

The mutated region of intercross (B3F1) lines between indaca rice (Kasarasu) and pyl-v mutant of Taichung No. 65 (T-65) was amplified by PCR. In the PCR, the oligonucleotides of SEQ ID NOs: 10 (F) and 11(R) for nDart1-0 (chromosome No. 3), those of SEQ ID NOs: 12 (F) and 13 (R) for nDart1-4 (3-1) (chromosome No. 3), those of SEQ ID NOs:14 (F) and 15 (R) for nDart1-12 (chromosome No. 1), those of SEQ ID NOs: 16 (F) and 17 (R) for nDart200-2 (chromosome No. 12) were used as primers.

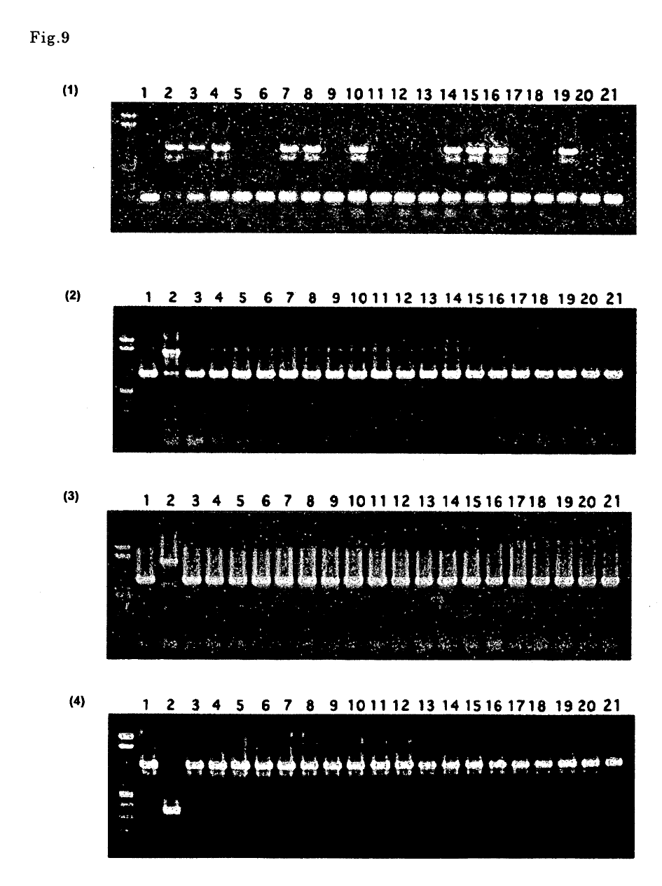

FIG. 9 shows the result of gel electrophoresis of the mutated region of the intercross lines (B3F1, FIG. 9, Line 3˜12). nDart (SEQ ID NO: 1) was transduced to 9 out of 19 individuals (FIG. 9 (1)). Since Kasarasu type bands are appeared at the same time, deletion of nDart is ongoing even in backcross individuals. Said nDart is located on the chromosome No. 3. Examining the extent of the transduction of Kasarasu on other chromosomes, it was found that the chromosome markers of chromosome No. 1, 3 and 12 are changed to Kasarasu type in all 19 individulas (FIG. 9, (2)-(4)). From the above results, it was concluded that nDart and an autonomous element (Dart) could keep their activity even in indica rice.

Furthermore, indica rice (93-11) was treated with azacytidine and confirmed release of nDart. Under usual growth conditions, release of nDart could not be observed, but the release was confirmed in 93-11 treated with azacytidine.

TABLE 1

| Element | TSD | TIR(bp) | 5′ TIR (5′->3′) | 3′TIR (5′->3′) | Size | TP | Reference |
|---|---|---|---|---|---|---|---|
| Ac | 8 | 11 | CAGGGATGAAA | TTTCATCCCTA | 4565 | + | Müller-Neumann |
| | | | | | | | et al. (1984) |
| As5145 | 8 | 11 | CAGGGATGAAA | TTTCATCCCTc | 4565 | −3800 | Xiao and |
| | | | | | | | Peterson (2002) |
| Ds1/rUq | 8 | 11 | CAGGGATGAAA | TTTCATCCCTA | 401-406 | | Gerlach et al. |
| | | | | | | | (1987) |
| Ds(sh-m5933) | 8 | 11 | TAGGGATGAAA | TTTCATCCCTA | 2040 | + | Doring et al. |
| | | | | | | | (1984) |
| Tam3 | 8 | 12 | TAAAGATGTGAA | TTCACATCTTTA | 3629 | + | Hehl et al. |
| | | | | | | | (1991) |
| nDart SEQ ID NO: 1 | 8 | 19 | TAGAGGTGGCCAAACGGGC | GCCCGTTTGGCCACCTCTA | 607 | + | |

TABLE 2
Appearance of mutable individuals by the treatment with azacytidine

| | Normal | | Individual with mutable or | | Germination rate | Mutable individual |
|---|---|---|---|---|---|---|
| | individual | Mutable individual | abnormal phenotype | Total | (%) | (%) |
| non-treated individual | 89 | 0 | 0 | 89 | 89 | 0.0 |
| azaC 0.15 mM | 39 | 43 | 17 | 99 | 99 | 60.6 |
| azaC 0.3 mM | 28 | 37 | 27 | 92 | 92 | 69.6 |
| azaC 0.45 mM | 25 | 34 | 36 | 95 | 95 | 73.7 |

TABLE 3

| | Nucleotide | | Conservation | Conservation | | |
|---|---|---|---|---|---|---|
| | sequence | Chromosome | of 5′ TIR (%) | of 3′ TIR (%) | TSD | Similarity to |
| nDart family | (bp) | No. | TAGAGGTGGCCAAACGGGC | GCCCGTTTGGCCACCTCTA | bp | nDart |
| nDart-d1 | 607 | 3 | 100% | 100% | 8 | 99.51% |
| nDart-d2 | 607 | 3 | ″ | ″ | 8 | 99.51% |
| nDart-d3 | 607 | 3 | ″ | ″ | 8 | 99.51% |
| nDart-d4 | 608 | 4 | ″ | ″ | 8 | 99.51% |
| nDart-d5 | 601 | 3 | ″ | ″ | 8 | 98.85% |
| nDart-d6 | 597 | 1 | ″ | ″ | 8 | 98.02% |
| nDart-l1 | 607 | Indica | ″ | ″ | 8 | 99.18% |

| | Site of substitution with nDart | | | | | | | | | | | | | |
|---|---|---|---|---|---|---|---|---|---|---|---|---|---|---|
| | (length from 5′ end) | | | | | | | Site of substitution with nDart | | | | | | |
| nDart family | 39 | 67 | 83 | 109 | 173 | 333 | 413 | 441 | 496 | 501 | 516 | 518 | 520 | 524 |
| nDart-d1 | C->T | | | | A->G | C->T | | | | | | | | |
| nDart-d2 | | | | G->A | A->G | | | | | | | | | G->A |
| nDart-d3 | | | | G->A | A->G | | | | | | | | | G->A |
| nDart-d4 | | | G->A | | A->G | | | | | A+ | | | | |
| nDart-d5 | | | | | A->G | | | CACGG- | | | | | | |

| | | | |
|---|---|---|---|
| nDart-d6 | CGGCACGGCC- | A->G | G->A |

| | | | | | |
|---|---|---|---|---|---|
| nDart-l1 | A->G | G->T | C->A | T->C | A->G |

1. A rice transposon gene comprising DNA of the following (1) or (2);
(1) DNA comprising the nucleotide sequence of SEQ ID NO: 1.
(2) DNA comprising the nucleotide sequence, which is more than 98% homologous to the nucleotide sequence of (1), wherein said DNA transposes by subjecting rice containing said DNA to the treatment with a chemical agent.

2. A rice transposon gene comprising DNA of the following (3) or (4);
(3) DNA comprising any nucleotide sequence of SEQ ID NO: 6-8.
(4) DNA comprising the nucleotide sequence, which is more than 98% homologous to the nucleotide sequence of (3), wherein said DNA transposes by subjecting rice containing said DNA to the treatment with a chemical agent.

3. The transposon gene as in claim 1, wherein said chemical agent is 5-azacytidine.

4. A plasmid comprising a transposon gene as in claim 1.

5. A transformant transduced a transposon gene as in claim 1.

6. A transformant as in claim 5, wherein the host comprises a plant.

7. A transformant as in claim 6, wherein the host is arabidopsis, tobacco, tomato, petunia, crucifer, cotton plant or maize.

8. A method for transposing a transposon gene comprising DNA of the following (1) or (2);
(1) DNA comprising the nucleotide sequence of SEQ ID NO: 1.
(2) DNA comprising the nucleotide sequence, which is more than 98% homologous to the nucleotide sequence of (1), wherein said DNA transposes by subjecting rice containing said DNA to the treatment with a chemical agent, which comprises treating a transformant as in claim 5 with a chemical agent.

9. The method as in claim 7, wherein said chemical agent is 5-azacytidine.

10. A transformed plant or seed, wherein said transposon gene is transposed by the method as in claim 8.

11. The transposon gene as in claim 2, wherein said chemical agent is 5-azacytidine.

12. A plasmid comprising a transposon gene as in claim 2.

13. A transformant transduced a transposon gene as in claim 2.

14. A transformant as in claim 13, wherein the host comprises a plant.

15. A transformant as in claim 14, wherein the host is arabidopsis, tobacco, tomato, petunia, crucifer, cotton plant or maize.

16. A method for transposing a transposon gene comprising DNA of the following (3) or (4);
(3) DNA comprising any nucleotide sequence of SEQ ID NO: 6-8.
(4) DNA comprising the nucleotide sequence, which is more than 98% homologous to the nucleotide sequence of (3), wherein said DNA transposes by subjecting rice containing said DNA to the treatment with a chemical agent, which comprises treating a transformant as in claim 13 with a chemical agent.

17. The method as in claim 15, wherein said chemical agent is 5-azacytidine.

18. A transformed plant or seed, wherein said transposon gene is transposed by the method as in claim 17.


SEQUENCE LISTING (17 sequences)

SEQ ID NO: 1 - DNA, 607 bp, Oryza sativa
tagaggtggccaaacgggccgggccaaaacgggcggcccgaggcacggcggaacctgtagccggcacggcccggcacggcctgctacagtaacgggccgtgccggcacggcacgagtagccgtgccgtgcttgggccgccggccgagcccgcgggccggcacggcacggcacagctactgtagtaagtccgcatctcatccttccgcaagtccgtatctcatccctcccaactgacggcccagcccgctagccgcctccgcaagtccgttgagcacccctcctagctgatggcccagcccgccagccacctccgcaagtccgcatcgcatccctccgcgccatttcggttcctgggccaaccgtgccccgtccacggcccatttttcattcacgggcccgtactgacacggcgggccacacgcatgccgtgccggcacgggcacggcccggccatccacgggccgtgcttgggccggcggctcggcacgtgggtcgggacggcacggcccgtttcatgagccgtgcctaacgggccgtgccgaaacgggccgtgccggacccgtgcccgtgccgtgccgggccgggccgcccgtttggccacctcta

SEQ ID NO: 2 - DNA, 20 bp, Artificial Sequence
  primer
taacgggtgtgtgtctggtg

SEQ ID NO: 3 - DNA, 22 bp, Artificial Sequence
  primer
gcaactgaaacccttacttgaa

SEQ ID NO: 4 - DNA, 20 bp, Artificial Sequence
  primer
gcggttgaagggctttaagg

SEQ ID NO: 5 - DNA, 20 bp, Artificial Sequence
  primer
catcctccacgggtccacca

SEQ ID NO: 6 - DNA, 3591 bp, Oryza sativa
tagaggtggccaaatgggccgggccaaacgggcggcccgaggcacggccgaacctgtagcaggcacggcccggcacggcctgctacagtaacgggccgtgccggcacggcacgagtagccgtgccgtgcttgggccgctggccgagcccgcgggccggcacggcacggcacggctactgtagcgggccggcacggcacggcccgcggcacggcacggcacggcacgccggcggcccgtcaggcgcggacagggcgggcggcggtcgaatgggaaggcgccacgtggcactaacggctatttgaccgttcaaatttgaaaataaccgttgggaggctaaaaaattcataaaaatttcgaaaaaattccaaaaaatctcaaatttcgccctataaatagggcatgaaccccagccatttctcctcatcccacactcctcatcttgtgctctcaagtgttttaagtgctctctttgttctcaagtgtgcattttttttgattttgacaaaatttgctcaaattttgtcaaaaatcaaaattagtttcgtagttcaacagtttgatcgcagaggtttgaagagctcgcagttggaaagatgtaagtaatattcaaatttgtgtattatttgtattgtgtttgtgaattcaataaatattcgaaaatttgtttatgtcggtttaaattttcagaatggatccgaactttccataccagtcgccgtcgttcaccttgggtgatttcgaccccaactacatgtcggggtttgatggtacctccggatcggctccaactccaccatctgtggaggaggtaccggttcatacggctgtcgttgaggaggtaccggttcaggcggagacagcttcggaaggattttccggaaccgcgagcggaagtgtttcgacacacaccggctcgaagagatcgagaacctccggtgtgtggcaaagcttcgatgagataaaggaaacatgccccgacggaagggaggtatcgaaagcccgttgtagaatatgtaggcaaattttatctgctcgttcttctggtggtacaggtcacctcaagcgccatgcggagtcgtgtgccaagaagcaaggaatacaactccggcagcagcaacttatggtaaacccagacggtacggtacacagttgggagtacgatcccatggttgctcgggaatctcttgtccggttaatcgccaggcaagatttacccctgaactttggggagtcccctgcttttgaacattacattcagcaatctcataaccctaggtttaaagctgtgagtaggcaaacatcaactagagatttagagaatgtttatcacaaggaagcaactgcacttaaggaactgtttagtacatgtactttctctgttagtgttacttcagatatatggagtagtagagctagagaggattatcttagcgtagttgttcattttgttgatgatgattggcaattacaaaagagagttttagggcttaggttaatagatgtctcacatacaggagaaaacatagctgaaagaattagggaagtaattaatgaatttaatcttgctgataaaatatttgctgtcaccctagataatgcatctgctaattctagggctattgaaatattgcaacctttattttgtgtgtatgctcaatcttttctactccatcagcgttgtgcatgtcatataattaatttgattgttaagactggcatgaagagggtaggtgaccacatcgatgctgttcgtcaagcaatcgcgtggttaactgcttctaacccgcggattgctgcatggaagaggttttgcaatgcggccggtgtgaaagctcgtaagtttgccaccgatgcagagcatcggtggaatgcaacgtatttaatgttaaaagttgttttaccttatagtagtttactttctgattttgttcagtcacgtggtggcccaagaaacagtgacgggtcttcagtactgaacgagcatgtttgggcaattgtccaaaaattttaccaatttctagaaactttttatgattgtactctaactttgtcacaagtttattatccaactgctaatataattttgcacaaccttcttgaaattgctactttatttaaagaatacgaaaatgatgacgttctaactgaacctgtctttcacatgaaacaaaaatatttgaaatattggaaaaatatacctatgttgtatgctcttgcttttgttttagatcctaggtgtaaattaaggggattgtctgctattttatcacttgttggagatactataggtgtagattatagttctttttatactgaggttagacgtaaattatatgaggtttttggaagatatgaagtaaagtttcaggaagttcgccagcagagaccccctcctatccccactacaggtaagaagaagatacagtggggtaggatttggggtggatcgtcttcaagttcaatccaaggtggtggcagttcgtcggctacaagtggagacgcctcttcgcatgttgtggccgaagagttgtccggttatttggacagcgacgccatccaccacgaagcacaagatttcaacgtcctcgggtggtggaatgaccacaagataacatatcctgtgctttcaaaactagcacgggatgtgttgacggtgcccgtgtcgacggtgtcctccgaatcggccttcagtctatgcggccgaatcatcgaagaccggaggacgactctgcgcagcgaccacgtcgaaatgctactaagcgttaaagactgggagcttgctcgacaacatgcccaatacactgcggacaaccaagaattggctgcccagttcgagcaactctacctggatccagaccaaccccagtagaattttgttagaagtagttctgacctttgagctgtactcttttctttgtcatggttttctcattttcccctatgagtttttacatgacaaagtttttaaagaggcagcatgtatcattgtatcctgtaatgatataaacatcaataaaggtcattactatttttaacaaattcttttgcaatattttcgcaagtgtggatttatctttaaattatttcaaaataatgaatcacaatctatatttttaaatttttcaacacaacaaaaaaataccattttttcttttttttaacattagcaaatcattactttttaaaaaaacttttatttccattttttaaataccattttttcattttttaacattagtaaatcattacttttttttaaacattttatttccatttttaattttttttttccttatacatttcctttgcttttttttaaaaaaaaaacactgtgcactacaggctggcgggctggcggcctgccttcacgggccgccgtgccccgaacggcccgtgggccgcgggcgtgccgtgccggcacgggcacggcccggccatccacgggccgtgcttgggccggcggctcggcacgtgggccggcacggcacggcccgtttcatcagccgtgcctaacgggccgtgccgaaacgggccgtgccggaaccgtgcccgtgccgggccgggccgtgccgcccgtttggacacctata

SEQ ID NO: 7 - DNA, 3843 bp, Oryza sativa
tatacctggccaaatgggccgtgccaagccgggccggcccaagcacgaccgcactgtagaaggcccaggcccggcacggcacgccggcctgtgggccgtgccggcacggcccgtttacccgtgccgtgtctgggccgacgccatagcccgtgggccagcacggcacggcacgtttactgtagagggctggcacggcccggcacggcccgcgagcacggcacggcacgggatcggctcggccgcgcacaccgcggcaccgcgcacgtggcgccaagcggccgagccgccgagggcagccgcggggccaggcggcgggaagcgcgcgtcgcttcgttcgcgcgtggcgcgtggcaagcggcgtcgcgacgtgtcggcgtgtcgctggggctggccagctgggaggctgggacgctgcctcacgctgggtcggtcgctcactcgctctcgctgcctgtctgcctgtctgccactctgcctccgtgcctcgttgggagcagccgagacagcgactcggcgagaccccgtacggcggccgaacggctagtcaaacgacgaatgcgagagtgccacgtgtcccccaacggctagtgagctaatccaacgaccagaagtagccgttggagagcaaaaaaaaaggaaaaaaattcgaaaaaaatatgaaatttatttctataaataggacacccaccagagcattctgaatcatccatacctcccattttgtgctctgttgtgctctttcgtgtgatagatcgattttttgatttagacaaaatttgacaaaattttgtgtaaaatcaaaaatagtttgtgactaaaaatagtgcatacaagaggtgtaaagccaagcaaaggtggtaagaaagaagtacgtcatatattcagttttatgtattattttattttatgttatgcgaataaatattctgaaatttgtttatgttgttttaaattttcagaatggatgaatcgaacattccatcgttcacgttaggtgatttcgaccctaactacgtgtcgaggtcattcccaactggtgagtatgatgccaccggatcggctccaacaccaccagttatggagccaccggcgggttcagaagcatccggcactatgagtgggagtgcatcgacgaacaccggctcaaagagatcaagaacttccggtgtttggcaacatttcgatgaggtggccatgacaggccctgatggaaggcaggtaacattcgcgagatgtagaatatgcaaaaataagttatctgcaaaatcatctggtggaacaggacatttgaagcggcatgccgaggcttgtgcaaagaagcaaggaatccaactacgacagcaacaactactactaaatcctgatggtacggtacgtacgtgggagtatgatcctatggtagctcgagaaaatcttgcccgtttaattgctagacaagatttacccttgaactttggtgagagtcctgcatttgaaaattacataaaaaattctcataatcctaggtttcaagctgttagtagacaaaccacaacccgtgatttgaaaaatgtctatgacaaaggttatgaatcactgaaggaattatttagtacatgcaccttttctgtcagtgtcacctcagacatatggagtagtagggctaaagaggattaccttagtgtagttgtacatttcattgatgatgattggcaaatgcaaaaaagagttcttggcttaaggttaattgatgtttcacatactggtgaaaatatagcagagagaattcgagaggttattgatgagtttaaccttgcagataaaatttttgctgtaacaatggataatgcatctgcaaattctagggccatggaaattctacaaccattattttgtatttatgctcaatcatttcttctgcatcagcgttgtgcatgccatatcattaatctaattgttaaatgtgggtttaagagagttaatgtacacatcgacgctgttcgtcaagcaatcacgtggttaactgcttcaaacccacggattgcacagtggaaaaggtattgttgtgcatcgggtgagcccccacgtaagtttttaaccgatgcagaccatcggtggaatgccacttattttatgttaaaggttgtattaccttacaaggatttacttactgttttccttcaaacacgtaatggcccaaaaaacagtgatggccagccaatactgactgatcatacctggcacattgttgaaaggttcaatcaatttcttgaaacgtttcatgactgtactcttctgttatctcaagtatattatccaacagctaatttaattttgcataatattcttgaaattgccactttgttgaaagagtatgaaaatgatgaccttttaatgcccgttgtctttaatatgaaacaaaaatatcttaaatattggaaagacatccccatgttgtattcttttgcatttattcttgatcctaggggaaaattacggggattcctcaatattctttcacttattggagatattattaatgttgattattctacctattatgctgatgtcaaaactaaattctatgaggtatttcgaaagtatgaattaaagtttcagggagatcgcttgcaaagacccccacctgtacctgcagcaggtaagaaaaaattacagtggagcagaatttggggcagttcatcttctagccatggtggtggtaccagttcatcagcagcaagtggggacgctagatcgcatggtcctgccgaagagttgtccaactatttggatagcgatgccatcaggcatgaaacgtcagacttcaacgtactcgggtggtggaatgatcataagatgtcatatcctgtgctatcaaaactagcacgggatgtgttgacggtgcccgtatcttcggtatcctccgaatcagccttcagtctatgcggaagaattatcgaggataggagaacaagtctgagcagcgatcatgtggaaatactattaagcgtcaaagactgggaacttgctgcagaacatgcccaatacactgctgacaaccaagaattggccgcacagttcgaaaacctttatttagatgacgaacaattagggtagctagtttatattttttaagtattgacctgttggctgtactcttttctttgtcatggttttctcaaatatgagtttttacatgataaagtttttaacgaggcagcatgtatcatgtaaacatcaataaaggtcattactcttttttcctcatatttttctaatatttttctaagtctaattatttttctatttttctccaactatccattaattttctcttagcttagttaactttcagacctttctctttgatttgaattgttccactgacagagtgacagcctgacagtgacagactgacaggcaatagacacacggtgacggacagcgtcagcaagtccagcgccaccgccgccacgtgtcgcccttcggccggccggtcgcgcggccccggccgctcgctcccgcgtgccgcgttgaaaatttcagccgcgccgcgcgcgcgccttgtcggcgactcggcgttgtcgcctagccgagtccttcggccgtgccgcgtgcccgcgtccttggctgcagtccgtcgtgccaacgggctgaccacggcccatgggccattgacgtgcccgtgccggcacggcacggcacgacgttccctcgggccgtgcttgggccggggagtaggcacgtgggccggcacggcacggcccgctataggagtcgtgcctaacgggccgtgccctagcgggccgtgccgccggcgtgcccgtgccgtgctgggccgggccgcccgtttggccaggtata

SEQ ID NO: 8 - DNA, 3732 bp, Oryza sativa
tatacctggccaaatgggccgtgccaagccgggccggcccaagcacgaccgcactgtagaaggcccaggcctggcacagcacgccggcctgtgggccgtgccggcacggcccgtttacccgtgccgtgtctgggccgacgccatagcccgtgggccagcacggcacggcacgtttactgtagagggctggcacggcatggcacggcccgcgagcacggcacggcacgggagcggcctagggtaggcacaccgcacacgtggcgccaagcggccgagccgccgagggcagccgcggggccaggcggcgggaagcgcgcgtcgctgcgttcgcgcgtggcgcgtggcaagcggcgtcgcgacgtgtcgctagggctgggaggctgggtcgctctcgctctgactgcctccgtcactccgtgcctcgttgggagcagccgagacggcgacaggcgactcagcgagaccccatacggcggccgaacagctagtcaaacgacgaatgcgagagtgccacgtgtccccaacggctagtgagctaatccaacgaccgctgtttttgagaagtagccgttggagagcaaaaaaatggaaaaaaattcgaaaaaaatatgaaatttatttctataaataggacacccaccggagcattctgaatcatctatacctcccattttgtgctctgttgtgctctttcgtgtgatagatcgattttttgatttagacaaaattttgtctaaaatcaaaaatagtttgtgactaaaaatagtgcatacaagaggtgtaaagccaagcaaaggtggtaagaaagaagtacgtcatatattcagttttatgtattattttattttatgttatgcgaataaatattctgaaatttgtttatgttgttttaaattttcagaatggacgaatcgaacattccatcgttcacgttaggtgatttcgaccctaactacgtgtcgaggtcattcccaactggtgagtatgatgccaccggatcggctccaacaccaccagttatggagccaccagcgggttcagaagcatccggcgctatgagtgggagtgcatcgacgaacaccggctcaaagagatcaagaacttccggtgtttggcaacatttcgatgaggtggccgtgacaggccctgatggaaggcaggtaacattcgcgagatgtagaatatgcaaaaataagttatctgcaaaatcatctggtggaataggacatttgaagcggcatgccgaggcttgtgcaaagaagcaaggaatccaactacgacagcaacaactactactaaatcctgatggtacggtacgtacgtgggagtatgatcctatggtagctcgagaaaatcttgcccgtttaattgctagacaagatttacccttgaactttggtgagagtcctgcatttgaaaattacataaaaaaattctcataatcctaggtttcaagctgttagtagacaaaccacaacccgtgatttgaaaaatgtctatgacaaaggttatgaatcactgaaggaattattaagtacatgcaccttttctgtcagtgtcacctcagacatatggagtagtagggctaaagaggattaccttagtgtagttgtacatttcattgatgatgattggcaaatgcaaaaaagagttcttggcttaaggttaattgatgtttcacatactggtgaaaatatagcagagagaattcgagaggttattgatgagtttaaccttgcagataaaatttttgctgtaacaatggataatgcatctgcaaattctagggccatggaaattctacaaccattattttgtatttatgctcaatcatttcttctgcatcagcgttgtgcatgccatatcattaatctaattgttaaatgtgggtttaagagagttaatgtacagatcgacgctgttcgtcaagcaatcacgtggttaactgcttcaaacccacggattgcacagtggaaaaggtattgttgtgcatcgggtgagcccccacgtaagtttttaaccgatgcagaccatcggtggaatgccatttattttatgttaaaggttgtattaccttacaaggatttacttactgttttccttcaaacatgtaatggcccaaaaaacagtgacggccagccaatactgactgatcatacctggcacattgttgaaaggttcaatcaatttcttgaaacgtttcatgactgtactcttctgttatctcaagtatattatccaacagctaatttaattttgcataatattcttgaaattgccactttgttgaaagagtatgaaaatgatgaccttttaatgcccgttgtctttaatatgaaacaaaaatatcttaaatattggaaagatatcctcatgttgtattcttttgcatttattcttgatcctaggggaaaattacggggattcctcaatattctttcacttattggagatattattaatgttgattattctacctattatgctgatgtcaaaactaaattctatgaggtatttcgaaagtatgaattaaagtttcagggagatcgcttgcaaagacccccacctgtccttgcagcaggtaagaaaaaattacagtggagcagaatttggggcggttcatcttctagccatggtggtggtaccagttcatcagcagcaagtggagatgctagatcgcatggtcctgccgaagagttgtccaactatttggatagcgatgccatcaggcatgaaacgtcagacttcaacgtactcgggtggtggaatgatcataagatgtcatatcctgtgctatcaaaactagcacgggatgtgttgacggtgcccgtatcttcggtatcctccgaatcagccttcagtctatgcggaagaattatcgaggataggagaacaagtctgagcagcgatcatgtggaaatactattaagcgtcaaagactgggaacttgctgcagaacatgcccaatacactgctgacaaccaagaattggccgcacagttcgaaaacctttatttagatgacgaacaattagggtagctagtttatattttttaagtattgacctgttggctgtactcttttctttgtcatggttttctcaaatatgagtttttacatgacaaagtttttaacgaggcagcatgtatcatgtaaacatcaataaaggtcattactcttttttccccatatttttctaatatttttctaagtctaattatttttctatttttctccaactatccattaattttctcttagcttagttaactttcggacctttctctttgatttgaattgttccactgacagagtgacaggcgatagacacacggacagaggcaagtcactgagtcagcattcagcaagtccagcgccacgtgtcgcccttcggccggccggtcccgcggccccggccgctcgctcccgcgtgccgcgtccaaattttcatccgcgcgctcgccttgtcggcgttgtcgccttgccagcttgcctgcagtcgatcgtgccaacgggccgaccacgacccatgggccattgacgtgcccgtgcaggcacggcacggcacgacgttccctcgggccgtgcttgggccggggagtaggcacgtgggccggcacggcacggcccgctacaggagtcgtgcctaacgggccgtgccctagcgggccgtgccgctggcgtgcccgtgccgtgctgggccgggccgggccgcccgtttggccaggtata

SEQ ID NO: 9 - DNA, 1186 bp, Oryza sativa
acactattctctcttcttcttaccaccctctcccggataagaggcgcaaccaaagcccccaccctcgcccaaaacccccacgagccgcggccatggcgaccaccaccaccaccccctcctcctctctcaccgcccctctcctccgcccgagctcgaacgcgaaccccgccccgagatctctgccgctcctcaggagccggaggtgcgctcgggccgtggcgaccgccgccgccgccgctggccacggggccgctcatcagaggagcgggatttggtcgatcagggatgatttggtggtgccgaggtcgccctacttccctgtggagtatgcgtcggggcaggaacgcgggccatcgcccatggtgatggagcggttccagagcgtcgtcagccagctcttccagcacaggattatccggtgtggtggacccgtggaggatgatatggcgaacatcatcgttgcccagctgctatatctcgatgccatcgatcctaacaaggatatcattatgtatgtgaattctcctggaggatcagtgacagctgggatggccatattcgacacgatgaagcatatcagacctgatgtttccacagtttgtattggacttgctgcaagtatgggagcttttctgcttagtgctgggacaaaagggaagcgatacagcttacctaactcaagaataatgatccatcaacctctcggaggagcccaaggacaagagactgatcttgagatccaggctaatgagatgctgcatcacaaggctaacctgaatggatacctagcataccacactgggcagcccctagataagatcaacgtagatactgaccgtgattacttcatgagcgcgaaggaggcaaaggagtatggtctaattgatggagttatcatgaatccccttaaagcccttcaaccgcttcctgcttctagttagccatggagtgctcaatctccacggagcattttttggttatcttttagaactgttattgcatccactgtttttattagcttggcaagatagttttgcgattccacaagcaaccacatcctgaggcttcaaagtttgtacaatacagatgtactactaggaggatatcttctgcgatgaatattgcaacttatttgatgtactattaggaggatatcttctgcgatgaatattgcaacttatttgat

SEQ ID NO: 10 - DNA, 20 bp, Artificial sequence
  primer
gataagaggcgcaaccaaag

SEQ ID NO: 11 - DNA, 20 bp, Artificial sequence
  primer
tggagccacgacagagtaga

SEQ ID NO: 12 - DNA, 20 bp, Artificial sequence
  primer
gcactggtaaaatcgcctgt

SEQ ID NO: 13 - DNA, 20 bp, Artificial sequence
  primer
aggtggattgcagccttatg

SEQ ID NO: 14 - DNA, 20 bp, Artificial sequence
  primer
gaaacgcaccgtttagcaat

SEQ ID NO: 15 - DNA, 20 bp, Artificial sequence
  primer
atttgagcacgaggaggaga

SEQ ID NO: 16 - DNA, 20 bp, Artificial sequence
  primer
ttatctgggagggtgcagac

SEQ ID NO: 17 - DNA, 21 bp, Artificial sequence
  primer
cgatttggtctttccgttaga